(19) United States
(12) Patent Application Publication
(10) Pub. No.: US 2021/0145811 A1
(43) Pub. Date: May 20, 2021

(54) TREATMENT OF CANCER WITH EXON 14 SKIPPING MUTATION(S) OR EXON 14 SKIPPING PHENOTYPE

(71) Applicant: Eli Lilly and Company, Indianapolis, IN (US)

(72) Inventor: Sau-Chi Betty YAN, Indianapolis, IN (US)

(21) Appl. No.: 17/163,234

(22) Filed: Jan. 29, 2021

Related U.S. Application Data
(62) Division of application No. 16/345,866, filed on Apr. 29, 2019.
(60) Provisional application No. 62/422,879, filed on Nov. 16, 2016.

(51) Int. Cl. A61K 31/4439; A61P 35/00

(52) U.S. Cl. CPC A61K 31/4439; A61P 35/00

(57) ABSTRACT

The present invention provides a method of treating cancer with tumors bearing MET exon 14 skipping or MET exon 14 skipping phenotype comprising administering to a patient in need of such treatment an effective amount of the compound of Formula (I), or a pharmaceutically acceptable salt thereof:

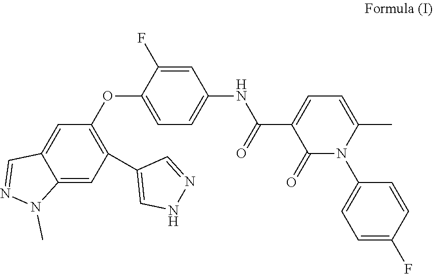 [SMILES: Cc1ccc(C(=O)Nc2ccc(Oc3cc4cnn(C)c4cc3-c3cn[nH]c3)c(F)c2)c(=O)n1-c1ccc(F)cc1]

### CROSS-REFERENCE TO RELATED PATENT APPLICATIONS

The present application is a divisional application of U.S. application Ser. No. 16/345,866, which application was filed on Apr. 29, 2019 and is the U.S. National Stage of International Application PCT/US2017/060796, filed on Nov. 9, 2017, which application claims the benefit of priority to U.S. Provisional Application No. 62/422,879, filed on Nov. 16, 2016, the contents of which applications are incorporated herein by reference in their entireties.

### BACKGROUND

The present invention relates to methods of using merestinib, or a pharmaceutically acceptable salt thereof, a type II MET kinase inhibitor, to treat certain disorders, such as lung cancer and gastric cancer, in patients with tumors bearing MET exon 14 skipping or MET exon 14 skipping phenotype.

Overexpression and activation of MET tyrosine receptor kinase can be an oncogenic driver of tumor growth in many types of cancer. The MET signaling pathway regulates a wide variety of normal cellular functions that can be subverted to support neoplasia, including cell proliferation, survival, apoptosis, scattering and motility, invasion, and angiogenesis. MET over-expression (with or without gene amplification), aberrant autocrine or paracrine ligand production, and missense MET mutations are mechanisms that lead to activation of the MET pathway in tumors and are associated with poor prognostic outcome.

Different genomic changes may occur in the intronic and/or exonic segments of MET and can lead to an alternatively spliced transcript of MET where exon 14 is skipped (i.e., exon 14 is largely or entirely deleted). MET exon 14 skipping mutations result in a protein missing the Y1003 phosphorylation site, the binding site for the ubiquitin ligase CBL, which targets MET for degradation. Additionally, a single point mutation at Y1003, D1002 or R1004 will also result in an inability of the ubiquitin ligase CBL to bind to the MET receptor without skipping of exon 14 (i.e., MET exon 14 skipping phenotype). This results in a MET protein with increased stability and oncogenic potential. MET exon 14 skipping mutations or an exon 14 skipping phenotype has been observed in adenosquamous, adenocarcinoma, sarcomatoid, squamous cell, large cell, and small cell histologies. Specifically, MET exon 14 skipping has been detected in lung adeonocarcinoma, as well as in neuroblastoma, gastric, and colon cancer cell lines. Tumors with MET exon 14 skipping mutations have been reported to be responsive to treatment with MET inhibitors in clinical case reports and series.

MET exon 14 skipping or MET exon 14 skipping phenotype is a targetable mutation in lung cancer and is reported in approximately 3 to 6% of non-small cell lung cancer (NSCLC) patients. Lung cancer remains the third most prevalent cancer in the United States and is the leading cause of cancer death in both men and women throughout the world. The two main types of lung cancer are small cell lung cancer and NSCLC. The majority of patients with lung cancer have advanced and/or metastatic disease at diagnosis and the majority of patients treated with curative intent develop recurrence. These patients present with advanced, inoperable stage cancer for which there is no prospect of cure. Treatment is provided to improve symptoms, optimize quality of life, and prolong survival.

MET exon 14 skipping or MET exon 14 skipping phenotype is also a targetable mutation in gastric cancer, a malignant tumor that originates in the stomach lining. Gastric cancers are classified according to the type of tissue from which they originate, with the most common type being adenocarcinoma and accounts for over 90% of all stomach cancers. Adenocarcinoma of the esophagus including carcinoma of the gastroesophageal junction (GEJ) is one of the fastest rising malignancies and is associated with a poor prognosis. Other forms of gastric cancer include lymphomas and sarcomas. Gastric cancer may be cured if it is found and treated at an early stage, but unfortunately, it is often found at a later stage.

There remains a need for the treatment of cancers with tumors bearing MET exon 14 skipping or MET exon 14 skipping phenotype. Tumors bearing MET exon 14 skipping or MET exon 14 skipping phenotype can have a response to MET inhibitors. Thus, merestinib, or a pharmaceutically acceptable salt thereof, may provide a treatment option for cancer patients who have tumors bearing MET exon 14 skipping or MET exon 14 skipping phenotype.

N-(3-Fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide (CAS #1206799-15-6), also known as merestinib, represented by the structural formula (I) below, is a small molecule type II MET kinase inhibitor. Merestinib and methods of making and using this compound and pharmaceutically acceptable salt(s) thereof including for the treatment of neoplastic diseases such as solid and non-solid tumors are disclosed in WO 2010/011538. Furthermore, merestinib is currently being evaluated in Phase 2 clinical studies for patients in NSCLC and solid tumors (see ClinicalTrials.gov NCT02920996).

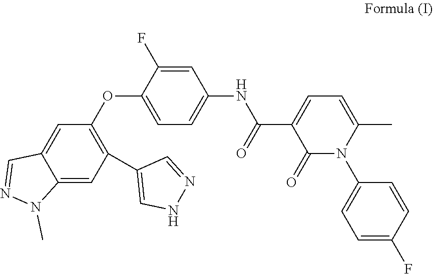 [SMILES: Cc1ccc(C(=O)Nc2ccc(Oc3cc4cnn(C)c4cc3-c3cn[nH]c3)c(F)c2)c(=O)n1-c1ccc(F)cc1]

### SUMMARY

Accordingly, the present invention provides a method of treating cancer with tumors bearing MET exon 14 skipping or MET exon 14 skipping phenotype, comprising administering to a patient in need of such treatment an effective amount of the compound of Formula (I), or a pharmaceutically acceptable salt thereof. Preferably, the cancer is lung, neuroblastoma, gastric, or colon cancer. More preferably, the cancer is gastric or lung cancer. Even more preferably, the cancer is lung cancer. Most preferably, the cancer is NSCLC.

Further, the present invention provides the use of a compound of Formula (I), or a pharmaceutically acceptable salt thereof, in therapy, in particular for treating cancer with tumors bearing MET exon 14 skipping or MET exon 14 skipping phenotype comprising administering to a patient in need of such treatment an effective amount of the compound of Formula (I), or a pharmaceutically acceptable salt thereof. Preferably, the cancer is lung, neuroblastoma, gastric, or colon cancer. More preferably, the cancer is gastric or lung cancer. Even more preferably, the cancer is lung cancer. Most preferably, the cancer is NSCLC. In a further embodiment, the present invention provides the use of a compound of the invention for the manufacture of a medicament for treating cancer with tumors bearing MET exon 14 skipping or MET exon 14 skipping phenotype. Preferably, the cancer is lung, neuroblastoma, gastric, or colon cancer. More preferably, the cancer is gastric or lung cancer. Even more preferably, the cancer is lung cancer. Most preferably, the cancer is NSCLC.

### DETAILED DESCRIPTION

In some embodiments of the present invention, the cancer patients are selected for treatment disclosed herein on the basis of having a tumor with MET exon 14 skipping mutations or MET exon 14 phenotype. Preferably, the MET exon 14 skipping mutation status of a cancer patient's tumor is determined by next generation gene sequencing methodologies. More preferably, the MET exon 14 skipping mutations or MET exon 14 phenotype of a cancer patient's tumor is determined by using Hybridization-captured Next Generation Sequencing (see, e.g., Schrock, A. B., et al., J Thoracic Oncology 2016, 9(11): 1493-1502). More preferably, the MET exon 14 phenotype of a cancer patient's tumor is determined by using the nCounter Analysis System (NANOSTRING® Technologies), a fluorescence-based platform for multiplexed digital mRNA profiling without amplification or generation of cDNA (see, e.g., Geiss, G. K., et al., Nature Biotechnology 2008, 26: 317-325).

As used herein, the terms “treating,” “to treat,” or “treatment” refers to restraining, slowing, stopping, reducing, or reversing the progression or severity of an existing symptom, disorder, condition, or disease.

As used herein, the term “patient” refers to a mammal, preferably a human.

As used herein, the terms “cancer” and “cancerous” refer to or describe the physiological condition in patients that is typically characterized by unregulated cell proliferation. Included in this definition are benign and malignant cancers. By “early stage cancer” or “early stage tumor” is meant a cancer that is not advanced or metastatic or is classified as a Stage 0, I, or II cancer. Examples of cancer include, but are not limited to, gastric cancer, preferably, carcinoma of the gastroesophageal junction, and lung cancer, preferably NSCLC.

The phrase “MET exon 14 skipping mutation”, “exon 14 skipping mutation”, “MET exon 14 skipping”, “exon 14 skipping”, or grammatical versions thereof, as used herein, refer to somatic mutations in the gene for MET, which, upon translation of the mRNA transcripts expressed thereby, result in cellular expression of MET polypeptides wherein exon 14 is largely or entirely deleted.

The phrases “MET exon 14 skipping phenotype”, “exon 14 skipping phenotype”, or grammatical versions thereof, as used herein refer to any single somatic point mutation in the gene for MET which, upon translation of the mRNA transcripts expressed thereby, result in cellular expression of MET polypeptides mutated at Y1003, D1002 or R1004 and which have a diminished ability to bind the ubiquitin ligase CBL, resulting in a MET protein with increased stability and oncogenic potential.

As used herein, the term “effective amount” refers to the amount or dose of compound of Formula (I), or a pharmaceutically acceptable salt thereof, upon administration to the patient, provides the desired effect in the patient under diagnosis or treatment. In determining the effective amount for a patient, a number of factors are considered by the attending diagnostician, including, but not limited to the patient's size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The compound of Formula (I) and its pharmaceutically acceptable salt(s) are generally effective over a broad dosage range. For example, dosages per day of individual agents normally fall within the range of about 60 mg/day to about 160 mg/day, preferably about 80 mg/day to about 160 mg/day, about 120 mg/day to about 160 mg/day. Most preferably, dosages per day of individual agents normally fall within the range of about 80 mg/day to about 120 mg/day. Most preferably the compound of Formula (I) is used at a dose per day selected from 60 mg, 80 mg, 120 mg, and 160 mg per day. Most preferably, the compound of Formula (I) is used at a dose per day selected from 80 mg and 120 mg.

### Example 1

### Evaluation of the Single Agent Merestinib (LY2801653) in a Xenograft Tumor Model Bearing MET Exon 14 Skipping

To determine the efficacy of merestinib in an Hs746t-derived xenograft mouse model of human gastric carcinoma, studies conducted essentially as described below may be performed. Hs746t is a gastric cancer cell line known to have MET exon 14 skipping and MET amplification (Asaoka et al., Biochem Biophys Res Commun 2010, 394:1042-1046).

### Study Designs and Methods:

### In Vitro Assay: Western Blotting

Hs746t cells are obtained from ATCC® (Manassas, Va.) and are maintained in DMEM Medium with L-glutamine and 10% fetal bovine serum (FBS). MKN45 cells, expressing wild-type MET, are obtained from JCRB Cell Bank (Japan) and are maintained in RPMI 1640 Medium with L-glutamine, 10% FBS, and sodium pyruvate. Cells are grown at 37° C. with 5% CO₂. Cells are seeded into 6-well plates, 1 million cells/well, and incubated overnight. Cells are incubated with merestinib for 2 hours, then are lysed in radioimmunoprecipitation assay (RIPA) buffer containing protease inhibitors. Protein concentrations of cell lysates are measured with the DC™ Protein Assay (BioRad), following manufacturer directions. Lysates are electrophoresed on Novex® 4-20% Tris Glycine gels (Invitrogen), and are transferred onto polyvinylidene difluoride (PVDF) membranes. The blots are probed for total MET (clone D1C2, CELL SIGNALING TECHNOLOGY®, Cat #8198), phospho-MET (Y1234/1235, clone D26, CELL SIGNALING TECHNOLOGY®, Cat #3077), and phospho-MET (Y1003, clone 13D11, CELL SIGNALING TECHNOLOGY®, Cat #3135). Monoclonal Anti-β-Actin (clone AC-15, SIGMA-ALDRICH®, Cat #A5441) is used as a loading control. After incubating with horseradish peroxidase (HRP)-linked secondary antibodies, blots are developed with chemiluminescent substrate and imaged on a Lumi-Imager (Roche).

### In Vitro Assay: Cell Proliferation Assay

Hs746t cells are seeded onto poly-D-lysine, 96-well plates, 3000 cells/well and allowed to attach overnight in a 37° C. with 5% CO₂ incubator. Merestinib is serially diluted 1:3 and added to the cells in triplicate. After 120 hours, cell viability is measured with the CELL TITER-GLO® Luminescent Cell Viability Assay (Promega), following manufacturer directions. The data are analyzed with GraphPad Prism v6 software. The assay is performed in duplicate experiments.

### In Vivo Hs746t Xenograft Model

Female athymic nude mice (Envigo) are used for this study. Food and water are available ad libitum. Animals are acclimated for 1 week prior to any experimental manipulation. The study is performed in accordance with AAALAC accredited institutional guidelines.

Merestinib is formulated as a solution in 10% PEG 400/90% (20% Captisol in water). Solution is freshly prepared every 7 days.

Hs746t cells are expanded in culture, harvested, and washed in Hank's Balanced Salt Solution (HBSS, GIBCO®). Approximately 5×10⁶ cells in HBSS are implanted subcutaneously into the hind flank of the animal. When tumors reach an average size of 150 to 200 mm³, the animals are randomized into groups of 7. Merestinib is prepared and administered via oral gavage at 6 or 12 mg/kg doses on a once daily schedule for 21 days.

Animals are sacrificed using CO₂ and cervical dislocation when tumors grew larger than 2000 mm³.

### Statistical Analysis

Tumor volumes and body weight are measured bi-weekly. Statistical analysis is performed when 3 of the 7 vehicle treated animals hed been removed from the study due to tumor burden. Tumor volume is transformed to the log scale to equalize variance across time and treatment groups. The log volume data are analyzed with a two-way repeated measures analysis of variance by time and treatment using the MIXED procedures in SAS software (Version 9.3). The correlation model for the repeated measures is spatial power. Treated groups are compared to the control group at each time point. The MIXED procedure is also used separately for each treatment group to calculate adjusted means and standard errors at each time point. Both analyses account for the autocorrelation within each animal and the loss of data that occurs when animals are removed or lost before the end of the study. The adjusted means and standard errors are plotted for each treatment group versus time.

Measure tumor growth with calipers. Calculate tumor volumes by the formula Volume (mm³)=L×W² (π/6) where L represents the larger diameter and W the smaller diameter. Calculate T/C % using the formula T/C %=100×ΔT/ΔC. Where ΔT=mean tumor volume of the drug-treated group on the final day of the study−mean tumor volume of the drug-treated group on the initial day of the dosing and ΔC=mean tumor volume of the control group on the final day of the study−mean tumor volume of the control group on the initial day of the dosing. Calculate changes in body weight by the formula (Weight on observation day−Weight on day 12)/Weight on Day 12×100. Calculate test for significant differences between treatment groups by RM ANOVA using the JMP (v.9.0.3) statistical package (SAS Institute Inc., Cary, N.C., USA).

### Results and Discussion:

The Hs746t gastric cancer cell line carries a homozygous genomic splicing mutation in MET at intron 14+1 G>T resulting in skipping of exon 14 in the mature mRNA (Asaoka et al., Biochem Biophys Res Commun 2010, 394:1042-1046). The MET mutant allele is also highly amplified. Western blots performed on lysates from in vitro cultured cells confirm a strong band corresponding to MET protein that migrates slightly faster than the corresponding band from MKN45 cells, expressing wild-type MET, indicating a protein of smaller size. Both cell lines express phosphorylated MET at the Y1234/1235 position (outside of exon 14), that is inhibited by merestinib treatment. However, Hs746t does not express phosphorylated MET at Y1003 (residing in exon 14), confirming the deletion of MET exon 14 at the RNA level. The effect of merestinib on in vitro Hs746t cell proliferation, as measured by the CELL TITER-GLO® assay after 5 days exposure, indicates an IC₅₀ of 33.4 nM (n=2) for merestinib.

In this study, merestinib is evaluated for anti-tumor effect in an Hs746t-derived mouse xenograft model. This model has a high level of tumor growth variance in the control group. In the vehicle control group (n=7), a tumor from one animal shows spontaneous regression, and one animal had to be removed due to tumor volume exceeding 2000 mm³ before the end of the study.

Merestinib at the 6 mg/kg dose initially causes tumor regression; however, tumors begin to increase in size beginning the tenth day after dosing began. At the end of the study, 5 of the 7 tumors show signs of regrowth (2 consecutively larger tumor volume measurements); however, anti-tumor activity (T/C=18.3%) is still significantly different than vehicle (p=0.033). Treatment with merestinib at the 12 mg/kg dose results in continual tumor regression (91.8%) to the end of the study. At this dose, 6 of the 7 animals are shown to be complete responders with 64 days of dosing. No tumor re-growth is observed indicating no treatment resistance within 2 months of treatment. After treatment is terminated, no tumor re-growth was observed for 5 weeks, indicating that these animals are complete responders.

There is significant differences in body weight in the groups treated with merestinib as compared to the vehicle control group; however, there are no noticeable health issues in any of the animals. Some of the weight difference may be attributed to the large tumor volumes in the vehicle group.

Taking all of these results in totality, the effect of merestinib in the Hs746t xenograft model results in significant tumor regression throughout treatment.

We claim:

1. A method of treating cancer with tumors bearing MET exon 14 skipping or MET exon 14 skipping phenotype comprising administering to a patient in need of such treatment an effective amount of the compound of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof.

2. The method according to claim 1 wherein the cancer is gastric or lung cancer.

3. The method according to claim 1 wherein the cancer is lung cancer.

4. The method according to claim 1 wherein the cancer is non-small cell lung cancer.

5. The use of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, in therapy.

6. The use of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, for treating cancer with tumors bearing MET exon 14 skipping or MET exon 14 skipping phenotype.

7. The use of N-(3-fluoro-4-(1-methyl-6-(1H-pyrazol-4-yl)-1H-indazol-5-yloxy)phenyl)-1-(4-fluorophenyl)-6-methyl-2-oxo-1,2-dihydropyridine-3-carboxamide, or a pharmaceutically acceptable salt thereof, for the manufacture of a medicament for treating cancer with tumors bearing MET exon 14 skipping or MET exon 14 skipping phenotype.

8. The use according to claim 5 wherein the cancer is gastric or lung cancer.

9. The use according to claim 5 wherein the cancer is lung cancer.

10. The use according to claim 5 wherein the cancer in non-small cell lung cancer.
